(19) United States
(12) Patent Application Publication
(10) Pub. No.: US 2026/0090770 A1
(43) Pub. Date: Apr. 2, 2026

(54) INFORMATION PROCESSING DEVICE, MEDICAL IMAGING APPARATUS, INFORMATION PROCESSING METHOD, AND INFORMATION PROCESSING PROGRAM

(71) Applicant: FUJIFILM Corporation, Tokyo (JP)

(72) Inventor: Hirohisa IZUMO, Tokyo (JP)

(73) Assignee: FUJIFILM Corporation, Tokyo (JP)

(21) Appl. No.: 19/332,088

(22) Filed: Sep. 18, 2025

(30) Foreign Application Priority Data
Sep. 27, 2024 (JP) 2024-169515

(51) Int. Cl. A61B 5/00; G06V 40/10

(52) U.S. Cl. CPC A61B 5/741; A61B 5/742; G06V 40/10; G06V 2201/03

(57) ABSTRACT

A console (information processing device) includes a recognition unit that recognizes an imaging period in which a part of a subject, which is associated with a presentation condition for presenting a predetermined instruction guide, is imaged from captured images obtained in time series by imaging the subject, and a presentation unit that presents the instruction guide in accordance with the recognized imaging period.

### CROSS-REFERENCE TO RELATED APPLICATIONS

This application claims priority under 35 USC 119 from Japanese Patent Application No. 2024-169515 filed on Sep. 27, 2024, the disclosure of which is incorporated by reference herein.

### BACKGROUND

### 1. Technical Field

The present disclosure relates to an information processing device, a medical imaging apparatus, an information processing method, and an information processing program.

### 2. Description of the Related Art

In recent years, with the advancement of medical apparatuses such as a computed tomography (CT) apparatus and a magnetic resonance imaging (MRI) apparatus, three-dimensional images having a higher resolution and a higher quality have been used for image diagnosis.

In the related art, the following technology has been used as a technology that can be applied to suppress a decrease in a quality of a captured image during an imaging period of a part of an organ or the like as a purpose of imaging for a subject in a case in which imaging is continuously performed on the subject by an imaging apparatus in a CT apparatus, an MRI apparatus, or the like.

JP2007-111255A discloses an X-ray CT apparatus for enabling main imaging at an appropriate timing in capturing a tomographic image using a contrast agent.

In this X-ray CT apparatus, a monitoring region of a subject to be subjected to monitoring imaging, a threshold value for starting auto voice, and a threshold value for starting imaging are set, a monitoring CT value (a concentration value of a contrast agent in a tomographic image) is acquired after starting the monitoring imaging, the auto voice is started in a case in which the monitoring CT value reaches the threshold value for starting the auto voice, and the main imaging is started in a case in which the monitoring CT value reaches the threshold value for starting the imaging.

JP2003-245275A discloses an X-ray computed tomography apparatus for reducing a deviation in a start timing of a main scan.

The X-ray computed tomography apparatus is an X-ray computer tomography apparatus that measures a start timing of a main scan by monitoring a contrast agent concentration administered to a subject by a preparatory scan, and includes a speaker that outputs guidance to the subject, and a real preparatory controller that supplies a voice signal to the speaker to output voice guidance in a case in which a CT value or a contrast agent concentration of tomographic image data obtained by the preparatory scan has reached or has exceeded a guidance threshold value, and starts execution or preparation of execution of the main scan in a case in which the CT value or the contrast agent concentration has reached or has exceeded a main scan threshold value.

JP2012-070890A discloses an X-ray CT apparatus for enabling an operation to be safely performed without increasing a burden.

The X-ray CT apparatus includes a moving body detection unit that detects whether there is a moving body in an examination room in which a gantry is disposed, a first voice output unit that outputs first voice, and a second voice output unit that outputs second sound, in which the X-ray CT apparatus performs monitoring imaging on a subject to which a contrast agent has been administered based on a first irradiation condition, obtains a CT value, which changes over time, of a region of interest on image data generated by the imaging, outputs the first voice in a case in which the CT value of the region of interest is a first CT value and there is the moving body in the examination room, and avoids outputting the voice in a case in which the CT value of the region of interest is the first CT value and there is no moving body in the examination room.

JP2009-189635A discloses an X-ray CT apparatus for reducing a burden on a subject in an examination of the subject by an X-ray CT apparatus, eliminating the complication of the examination by omitting a breathing instruction from an operator, and preventing a situation in which an image quality of an X-ray CT image deteriorates due to a failure in breath holding.

The X-ray CT apparatus sets, on an imaging range setting screen, an output position of previous voice to a position after a position at a start time point of imaging and an output position of subsequent voice between a position at an end time point of imaging and the output position of the previous voice, executes an instruction to hold breathing in a case in which the output position of the previous voice is detected, and executes an instruction to release the breathing holding in a case in which the output position of the subsequent voice is detected.

### SUMMARY

In the related art, in a case in which a plurality of parts such as a lung field and a lumbar vertebra are imaged by a single continuous imaging operation, an imaging sequence indicating a procedure of the imaging is set for each part that is an imaging target, and the imaging sequence is set in association with a voice guide. Therefore, in a case in which the voice guide such as “Please hold your breath” or “Please relax” is provided for each part that is an imaging target while the imaging is being performed, the voice guide associated with the imaging sequence set for the corresponding part may be sequentially reproduced.

On the other hand, in a case in which the plurality of parts are continuously imaged in a single imaging operation, it is desired that each part can be imaged in one imaging sequence.

However, in this case, although it is possible to reproduce the voice guide at the start or end of the imaging sequence, there is a problem in that it is difficult to reproduce the voice guide related to the part, for the part to be imaged next, in the middle of the imaging sequence in an accurate period. This is because there is no trigger to reproduce the corresponding voice guide in the middle of the imaging sequence.

On the other hand, in the technologies disclosed in JP2007-111255A, JP2003-245275A, JP2012-070890A, and JP2009-189635A, a period in which the voice guide is reproduced is determined in accordance with the concentration value of the contrast agent, a position of a table on which the subject lies, and the like, and the configuration cannot be applied to the technology of reproducing the voice guide in accordance with the imaging sequence. It should be noted that this problem may occur not only in a case in which the voice guide is reproduced, but also in a case in which various instruction guides are presented to the subject by display on a display unit, printing by an image forming device, or the like. In addition, this problem may occur not only in a case in which imaging is performed using the imaging sequence, but also in a case in which an imaging timing is determined using other preset information related to imaging.

The present disclosure has been made in view of the above-described circumstances, and an object of the present disclosure is to provide an information processing device, a medical imaging apparatus, an information processing method, and an information processing program that can present an instruction guide in an appropriate period even in a case in which imaging of a plurality of parts is performed by single continuous imaging.

A first aspect of the technology of the present disclosure relates to an information processing device comprising: a processor, in which the processor recognizes an imaging period in which a part of a subject, which is associated with a presentation condition for presenting a predetermined instruction guide, is imaged from captured images obtained in time series by imaging the subject, and presents the instruction guide in accordance with the recognized imaging period.

A second aspect of the technology of the present disclosure relates to the information processing device according to the first aspect, in which the imaging period is a period including at least one of a start time of an imaging sequence indicating a procedure of the imaging, an end time of the imaging sequence, or any time point in a period after the start time and before the end time.

A third aspect of the technology of the present disclosure relates to the information processing device according to the first or second aspect, in which the processor determines whether the imaging period is likely to be approaching from the captured images, and presents the instruction guide in accordance with the determined imaging period.

A fourth aspect of the technology of the present disclosure relates to the information processing device according to the third aspect, in which the processor determines whether the imaging period is likely to be approaching by using imaging conditions set in advance in correspondence with the imaging for the subject.

A fifth aspect of the technology of the present disclosure relates to the information processing device according to the third aspect, in which the processor determines whether the imaging period is likely to be approaching by determining, from the part recognized by image recognition on the captured image, whether another part to be subsequently included in the captured image is likely to be imaged.

A sixth aspect of the technology of the present disclosure relates to the information processing device according to the fifth aspect, in which the processor further presents a cross section image corresponding to the captured image for which the imaging period is recognized, such that the part recognized by the image recognition has a different state from other parts.

A seventh aspect of the technology of the present disclosure relates to the information processing device according to the first or second aspect, in which the instruction guide is a voice guide by voice, and the processor reproduces the voice guide in accordance with the recognized imaging period.

An eighth aspect of the technology of the present disclosure relates to the information processing device according to the first or second aspect, in which the instruction guide is a display guide by display, and the processor displays the display guide in accordance with the recognized imaging period.

A ninth aspect of the technology of the present disclosure relates to a medical imaging apparatus comprising: the information processing device according to the present disclosure; and an imaging device that performs main imaging for a medical image while the instruction guide is presented by the information processing device.

A tenth aspect of the technology of the present disclosure relates to an information processing method executed by a processor, comprising: recognizing an imaging period in which a part of a subject, which is associated with a presentation condition for presenting a predetermined instruction guide, is imaged from captured images obtained in time series by imaging the subject; and presenting the instruction guide in accordance with the recognized imaging period.

An eleventh aspect of the technology of the present disclosure relates to an information processing program causing a computer to execute a process comprising: recognizing an imaging period in which a part of a subject, which is associated with a presentation condition for presenting a predetermined instruction guide, is imaged from captured images obtained in time series by imaging the subject; and presenting the instruction guide in accordance with the recognized imaging period.

According to the present disclosure, it is possible to provide the information processing device, the medical imaging apparatus, the information processing method, and the information processing program that can present a voice guide in an appropriate period even in a case in which imaging for a plurality of parts is performed by single continuous imaging.

### BRIEF DESCRIPTION OF THE DRAWINGS

FIG. 1 is a diagram showing a schematic configuration of a medical diagnostic apparatus according to an embodiment of the technology of the present disclosure.

FIG. 2 is a block diagram showing an example of functional configurations of a console according to the embodiment of the technology of the present disclosure.

FIG. 3 is a schematic diagram showing an example of a configuration of a voice information database according to the embodiment of the technology of the present disclosure.

FIG. 4 is a diagram showing a problem of the related art, and is a diagram showing an example of a reproduction state of a voice guide in a case in which a plurality of parts are imaged using a plurality of imaging sequences by a single continuous imaging operation.

FIG. 5 is a diagram showing a problem of the related art, and is a diagram showing an example of a reproduction state of the voice guide in a case in which the plurality of parts are imaged using one imaging sequence by a single continuous imaging operation.

FIG. 6 is a diagram showing the technology of the present disclosure, and is a diagram showing an example of the reproduction state of the voice guide in a case in which the plurality of parts are imaged using one imaging sequence by a single continuous imaging operation according to the technology of the present disclosure.

FIG. 7 is a diagram showing the technology of the present disclosure, and is a diagram showing an example of a reproduction method of the voice guide in a case in which the plurality of parts are imaged using one imaging sequence by a single continuous imaging operation according to the technology of the present disclosure.

FIG. 8 is a flowchart showing a flow of control of the medical diagnostic apparatus by the console according to the embodiment of the technology of the present disclosure.

FIG. 9 is a diagram showing an example of a display state of a cross section image according to the embodiment of the technology of the present disclosure.

FIG. 10 is a diagram showing an example of a display state of a captured image according to the embodiment of the technology of the present disclosure.

### DETAILED DESCRIPTION

Hereinafter, an example of an embodiment of the technology of the present disclosure will be described with reference to the accompanying drawings. Here, a case will be described in which the presentation by the reproduction of a voice guide is applied as the presentation of an instruction guide according to the technology of the present disclosure. In the drawings, the same or equivalent components and parts are denoted by the same reference numerals. Furthermore, dimensional ratios in the drawings are exaggerated for convenience of description and may be different from the actual ratios.

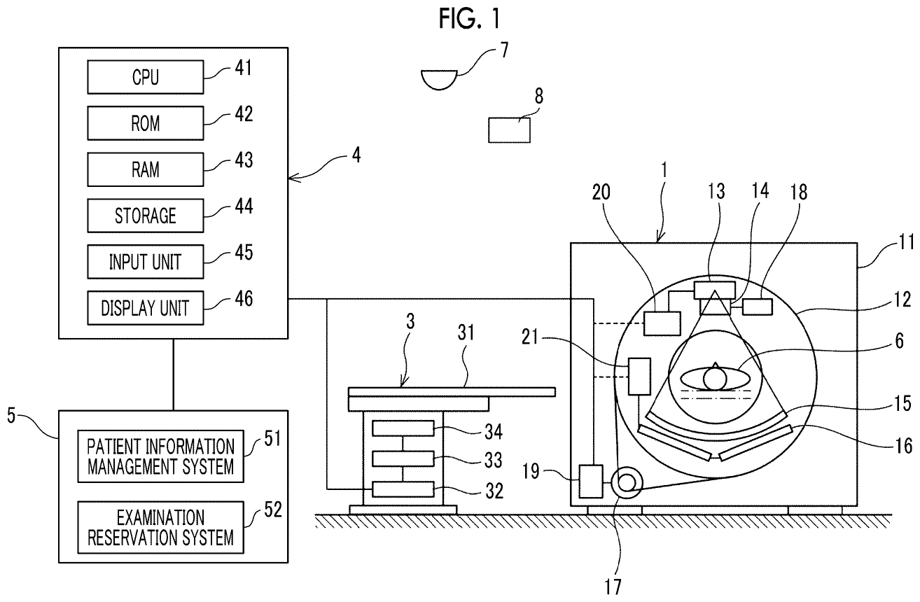

FIG. 1 is a diagram showing a schematic configuration of a medical diagnostic apparatus including an information processing device and a medical imaging apparatus according to the embodiment of the technology of the present disclosure. In the present embodiment, a configuration of an embodiment of an X-ray computed tomography (CT) apparatus as the medical diagnostic apparatus is shown. It should be noted that the medical diagnostic apparatus according to the present disclosure is not limited to the X-ray CT apparatus, and can be applied to other medical diagnostic apparatuses such as a magnetic resonance imaging (MRI) apparatus.

The medical diagnostic apparatus according to the present embodiment includes a scanner 1, an table 3, and a console 4. The console 4 corresponds to an information processing device according to the present disclosure, the scanner 1 corresponds to an imaging device according to the present disclosure, and a combination of the scanner 1, the table 3, and the console 4 corresponds to a medical imaging apparatus according to the present disclosure.

The scanner 1 is a unit that executes a CT scan. The scanner 1 comprises a gantry 11, a rotary plate 12 that has an opening at a center portion and is rotatably supported by the gantry 11, an X-ray tube device 13 fixed to the rotary plate 12, a collimator 14 provided in an X-ray emission port portion of the X-ray tube device 13, an X-ray detector 15 that is disposed to face the X-ray tube device 13 with the opening of the rotary plate 12 interposed therebetween, and a rotary plate drive device 17 provided in the gantry 11.

Furthermore, the rotary plate 12 of the scanner 1 comprises a collimator control device 18 that controls the collimator 14 to change an X-ray irradiation field, a rotary plate drive control device 19 that performs drive control of the rotary plate drive device 17, an X-ray high-voltage generation device 20 that supplies power for generating X-rays to the X-ray tube device 13 and controls X-ray generation conditions, a data collection device 16 that collects the output of the X-ray detector 15, and a data transmission device 21 that transmits the data collected by the data collection device 16. The supply of the power and the control signal to each unit provided in the rotary plate 12 and taking-out of the data from each unit provided in the rotary plate 12 are performed via a slip ring (not shown) provided between the gantry 11 and the rotary plate 12.

The table 3 is used for moving a subject between an imaging preparation position and an imaging position. The subject is placed on a top plate 31. Although not shown, an up/down movement mechanism and a front/rear movement mechanism of the top plate 31 are provided. In addition, the table 3 is provided with an table control device 32, a top plate up/down movement control device 33, and a top plate front/rear movement control device 34 in order to control operations of the up/down movement mechanism and the front/rear movement mechanism of the top plate 31.

The console 4 controls the medical diagnostic apparatus that is an X-ray CT apparatus. The console 4 is an example of a computer according to the embodiment of the present disclosure. The console 4 comprises a central processing unit (CPU) 41, a read-only memory (ROM) 42, a random-access memory (RAM) 43, and a storage 44. The console 4 may further comprise an input unit 45 and a display unit 46.

The CPU 41 that is an example of a processor is a central processing unit and executes various programs or controls each unit. That is, the CPU 41 reads out a program from the ROM 42 or the storage 44 to execute the program using the RAM 43 as a work area. The CPU 41 controls each of the above-described configurations and performs various types of arithmetic processing in accordance with the program recorded in the ROM 42 or the storage 44. In the present embodiment, a control program of the medical diagnostic apparatus, including information processing of determining an imaging range in main imaging by the X-ray CT apparatus, reproducing the voice guide, and the like, is stored in the ROM 42 or the storage 44. The program related to the information processing corresponds to an information processing program according to the present disclosure, and a processing method by the information processing corresponds to an information processing method according to the present disclosure.

The ROM 42 stores various programs and various data. The RAM 43, as the work area, temporarily stores the program or the data. The storage 44 is configured by a storage device such as a hard disk drive (HDD), a solid-state drive (SSD), or a flash memory, and stores various data and various programs including an operating system.

The input unit 45 includes a pointing device, such as a mouse, and a keyboard, and is used to perform various inputs. The input unit 45 comprises an table operation unit that operates a height of the table 3.

The display unit 46 is, for example, a liquid-crystal display and displays various types of information. The display unit 46 may employ a touch panel type, to function as the input unit 45.

In the present embodiment, the console 4 is connected to a hospital information system 5 of a hospital in which the X-ray CT apparatus is installed. The hospital information system 5 comprises a patient information management system 51 and an examination reservation system 52.

The patient information management system 51 is a database in which personal information, medical treatment data, examination image data, examination data, medication data, and the like of a patient who has received medical treatment in the hospital in the past are stored. The personal information of the patient includes personal identification information such as a name, an identification (ID), a date of birth, an age, and a gender, as well as physical information such as a height and a weight and a past medical examination date. These pieces of personal information are input to the system in a case in which the subject visits the hospital as the patient and receives medical treatment. The height and the weight of the patient are collected by the patient's self-reporting on a medical questionnaire or collected by actual measurement, and are input to the patient information management system 51. Further, the medical treatment data, the examination image data, the examination data, and the medication data are input to the patient information management system 51 each time the patient receives medical treatment, and an in-hospital patient database is created.

Meanwhile, the examination data includes information related to the examination of the patient, such as a non-organ part as an examination target, an organ as an examination target, and a scan protocol to be applied in the examination of the corresponding patient. Here, the non-organ part as the examination target is information indicating a part such as a head, a chest, an abdomen, and a thoracoabdominal region, and the organ as the examination target is information indicating an organ itself such as a lung field and a stomach. In addition, the scan protocol is information indicating setting conditions applied at the time of the main imaging, such as an imaging mode applied at the time of the examination, an mAs value set in the X-ray tube device 13, and an initial setting of the imaging range.

The examination reservation system 52 creates an examination schedule for the day for each system of the medical diagnostic apparatus equipped in the hospital based on the information stored or written in the patient information management system 51, and distributes the created examination schedule to each system as the data. The examination schedule includes an order of patients who undergo the examination on the day in the medical diagnostic apparatus and the personal identification information and the physical information of each patient. The content of the examination reservation system 52 is sequentially updated in accordance with the progress of the medical treatment of the day, and the updated data is transmitted to each system each time the content is updated.

A camera 7 is provided on a ceiling of an imaging room in which the medical diagnostic apparatus is provided, in a state in which a region within a movable range of the table 3 can be imaged. The camera 7 according to the present embodiment is assumed to be capable of obtaining an optical image (hereinafter, referred to as a “camera image”) within an imaging angle of view, by imaging. In the present embodiment, a camera that captures a color still image is applied as the camera 7, but the present disclosure is not limited to this form. For example, a form may be adopted in which the camera 7 that captures a monochrome still image is applied, or a form may be adopted in which the camera 7 that captures a color or monochrome moving image is applied.

In a case in which the control program of the medical diagnostic apparatus is executed, the console 4 implements various functions by using hardware resources described above. Functional configurations implemented by the console 4 will be described.

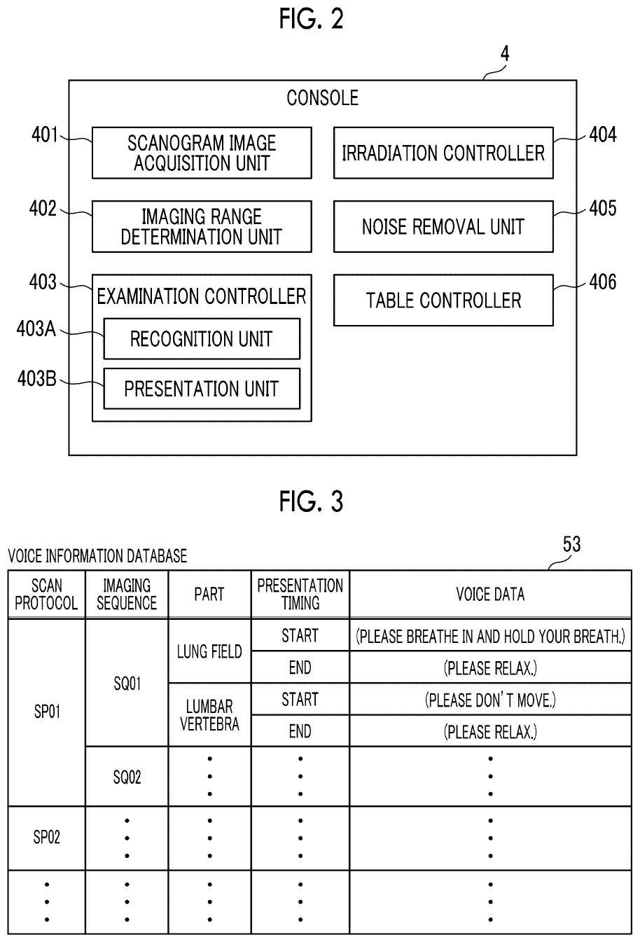

FIG. 2 is a block diagram showing an example of the functional configurations of the console 4.

As shown in FIG. 2, the console 4 has a scanogram image acquisition unit 401, an imaging range determination unit 402, an examination controller 403, an irradiation controller 404, a noise removal unit 405, and a table controller 406 as the functional configurations. Each functional configuration is implemented by the CPU 41 reading out and executing the control program of the medical diagnostic apparatus stored in the ROM 42 or the storage 44.

The scanogram image acquisition unit 401 executes processing of acquiring a scanogram image of the subject based on the irradiation of the subject with X-rays controlled by the irradiation controller 404 described later. The scanogram image can be obtained by detecting the X-rays transmitted through the subject with the X-ray detector 15. The scanogram image acquisition unit 401 acquires the scanogram image including at least a most protruding region of the abdomen (in the present embodiment, a central portion of the abdomen) of the subject. Although the scanogram image is also referred to as a scout image, a topogram, or the like, hereinafter, the scanogram image will be referred to as a “scanogram image”.

The imaging range determination unit 402 determines the imaging range in the main imaging using the scanogram image.

In the present embodiment, first, feature points in a contour of a part such as an organ or a bone part that is the examination target of the subject are recognized from the scanogram image, and a region surrounding each feature point is specified as a candidate for the imaging range for each of the same parts. Then, a circumscribing rectangular frame of the specified candidate imaging range or a region wider than the circumscribing rectangular frame by a predetermined margin amount is determined as a final imaging range. As described above, in the present embodiment, since the imaging range is determined in accordance with the circumscribing rectangular frame including an examination target part, in a case in which there are a plurality of examination target parts, a rectangular frame including all the candidates for a plurality of imaging ranges corresponding to the plurality of parts is a finally determined imaging range in the main imaging.

As described above, in the present embodiment, a case has been described in which the imaging range is determined using the scanogram image, but the present disclosure is not limited to this form. For example, a form may be adopted in which the imaging range is determined using the camera image obtained by imaging with the camera 7. In this case, for example, a form can be described in which feature points of parts included in the examination target part of the subject, such as both eyes, a nose, both shoulders, both hands, both hips, and both knees, from the camera image are recognized, and a range connecting the feature points including the examination target part is determined as the final imaging range at the time of the main imaging. In addition, a form may be adopted in which the scanogram image or the camera image is displayed, and the imaging range at the time of the main imaging is designated by a person in charge of imaging with respect to the displayed image.

The examination controller 403 controls an examination sequence of the subject by the medical diagnostic apparatus. As shown in FIG. 2, the examination controller 403 according to the present embodiment includes a recognition unit 403A and a presentation unit 403B.

The recognition unit 403A recognizes an imaging period in which a part of the subject, which is associated with a presentation condition for presenting a predetermined instruction guide, is imaged, from captured images corresponding to the medical images according to the present disclosure, which are obtained in time series by imaging the subject with the scanner 1. Then, the presentation unit 403B presents the instruction guide in accordance with the imaging period recognized by the recognition unit 403A. In a case in which the imaging is performed by the scanner 1, the above-described instruction guide represents information indicating an instruction for improving a quality of the captured image, such as “Please hold your breath” and “Please relax” to the subject.

In the present embodiment, a timing (hereinafter, referred to as a “presentation timing”) at which a corresponding instruction guide is presented is applied as the “presentation condition”, and a timing (hereinafter, referred to as an “imaging timing”) at which the part associated with the presentation timing is imaged is applied as the “imaging period”.

That is, the recognition unit 403A according to the present embodiment recognizes, from the captured images obtained in time series by imaging the subject, the imaging timing at which the part of the subject, which is associated with the presentation timing at which the predetermined instruction guide is presented, is imaged. Then, the presentation unit 403B according to the present embodiment presents the instruction guide in accordance with the recognized imaging timing.

As a result, for example, in a case in which a first timing at which imaging of the part associated with the presentation timing is started and a second timing at which the imaging is ended are applied as the presentation timings, the instruction guide is presented at the first timing and the second timing.

As described above, in the present embodiment, the “presentation timing” is applied as the “presentation condition”, and the “imaging timing” is applied as the “imaging period”, but the present disclosure is not limited to this. For example, a form may be adopted in which a condition for presenting the corresponding instruction guide, in which the body movement of the subject is stationary is applied as the “presentation condition”, and a stationary period of the body movement of the subject is applied as the “imaging period”. The phrase “body movement of the subject is stationary” here means not only that the body movement of the subject is completely stationary, but also a stationary state that tolerates a degree of the body movement of the subject such that the quality of the resulting captured image does not hinder the intended interpretation. Similarly, the phrase “stationary period of the body movement of the subject” referred to here means not only a period in which the body movement of the subject is completely stopped, but also a stationary period that tolerates a degree of the body movement of the subject such that the quality of the resulting captured image does not hinder the intended interpretation.

That is, in this form, the recognition unit 403A recognizes the stationary period of the body movement of the subject in which the part of the subject, which is associated with the condition for presenting the predetermined instruction guide, in which the body movement of the subject is stationary, is imaged from the captured images obtained in time series by imaging the subject. Then, the presentation unit 403B according to the present embodiment presents the instruction guide in accordance with the stationary period of the body movement of the recognized subject.

In the present embodiment, as the above-described imaging period, that is, as the above-described imaging timing, a timing (in the present embodiment, all the timings) including at least one of a start time of the imaging sequence indicating a procedure of the imaging, an end time of the imaging sequence, or a time point in a period after the start time and before the end time is applied.

In addition, the recognition unit 403A according to the present embodiment determines whether the imaging period, that is, the imaging timing is likely to be approaching from the captured images, and the presentation unit 403B presents the instruction guide in accordance with the imaging period determined by the recognition unit 403A. In the present embodiment, determination by predicting the approach of the imaging timing is applied as the determination of whether the imaging timing is likely to be approaching.

In addition, in the present embodiment, the determination of whether the imaging period is likely to be approaching, that is, the prediction of the approach of the imaging timing is performed by predicting, from the part recognized by image recognition on the captured image, whether another part to be subsequently included in the captured image is imaged.

Then, the presentation unit 403B according to the present embodiment further presents a cross section image corresponding to the captured image for which the imaging period, that is, the imaging timing is recognized, such that the part recognized by the image recognition has a different state from the other parts.

In the present embodiment, the voice guide by voice is applied as the instruction guide, and the presentation unit 403B according to the present embodiment presents the voice guide by reproducing the voice guide in accordance with the imaging period (imaging timing) recognized by the recognition unit 403A. Therefore, the imaging room according to the present embodiment is provided with a speaker 8 (see also FIG. 1) for reproducing the voice guide.

As described above, in the present embodiment, a case has been described in which the voice guide is applied as the instruction guide, and the voice guide is reproduced, but the present disclosure is not limited to this form. For example, a form may be adopted in which a display guide by display is applied as the instruction guide, and the presentation unit 403B presents the display guide by displaying the display guide in accordance with the recognized imaging period (imaging timing). In this case, the content of the instruction guide can be understood by the display.

Meanwhile, the irradiation controller 404 controls an irradiation amount of the X-rays from the X-ray tube device 13 to the subject. For example, although a shoulder requires a large dose, a lung is filled with air and thus has a small attenuation, so a small dose may be sufficient, and a liver has a large attenuation and thus requires a more dose. The irradiation controller 404 controls the irradiation amount of the X-rays to the subject in accordance with the part of the subject. A required image quality level is different between the scanogram image and an image (image of the main imaging) obtained by the examination, and the image of the main imaging is required to have a higher image quality than the scanogram image. Therefore, the irradiation controller 404 may obtain the irradiation amount of the X-rays to the subject by multiplying dose data in a case of the scanogram image by a coefficient or converting the dose data using a table. The irradiation controller 404 can obtain an examination image of the subject having a noise amount desired by an operator by controlling the irradiation amount of the X-rays to the subject using the dose data of the X-rays in a case in which the scanogram image is generated.

In a case in which the noise amount of the examination image obtained by the irradiation of the subject with the X-rays exceeds an allowable amount of a desired noise amount, the noise removal unit 405 performs noise removal (denoising) processing on the examination image. For example, in a case in which a dose modulation based on the dose data of the subject associated with a past scanogram image does not result in a desired image quality index (for example, an image noise) due to an increase in weight of the subject, and the image noise is increased, the noise removal unit 405 increases an intensity level of the denoising processing to suppress the image noise and automatically performs reconstruction to achieve a desired image quality indicator that is substantially the same as that in the past examination.

The table controller 406 controls the height of the table 3. The table controller 406 may control the height of the table 3 based on an operation of a technician, or may control the height of the table 3 based on data of a most recent examination of the subject.

Meanwhile, in the medical diagnostic apparatus according to the present embodiment, in order to realize the control of the examination sequence by the examination controller 403, a voice information database, in which data (hereinafter, referred to as “voice data”) indicating the voice guide is registered, is registered in the hospital information system 5.

Next, the voice information database according to the present embodiment will be described with reference to FIG. 3. FIG. 3 is a schematic diagram showing an example of a configuration of a voice information database 53 according to the present embodiment.

The voice information database 53 according to the present embodiment is a database in which the voice data indicating the voice guide described above is registered. As shown in FIG. 3, the voice information database 53 according to the present embodiment stores pieces of information of the scan protocol, the imaging sequence, the part, the presentation timing, and the voice data in association with each other.

The scan protocol is information indicating the scan protocol itself, and the imaging sequence is information indicating the imaging sequence described above, which is applied in the corresponding scan protocol. In addition, the part is information indicating a part to be targeted by the corresponding scan protocol and imaging sequence, and the presentation timing is information indicating a timing at which the corresponding voice data is reproduced in a case of imaging the corresponding part. The voice data is information indicating the voice data itself to be reproduced at the corresponding presentation timing.

In the example shown in FIG. 3, for example, in an imaging sequence SQ01 in a scan protocol SP01, it is registered that the examination target parts are “lung field” and “lumbar vertebra”. In this example, in a case in which the imaging is performed, information in which voice of “Please breathe in and hold your breath” is reproduced at a timing at which the imaging of the lung field is started and voice of “Please relax” is reproduced at a timing at which the imaging of the lung field is ended is registered for the lung field.

As described above, in the present embodiment, a case has been described in which the scan protocol, the imaging sequence, the part, the presentation timing, and the voice data are collectively configured as one database, but the present disclosure is not limited to this. For example, a form may be adopted in which the database is configured to be different in terms of the scan protocol and the imaging sequence, and the part, the presentation timing, and the voice data.

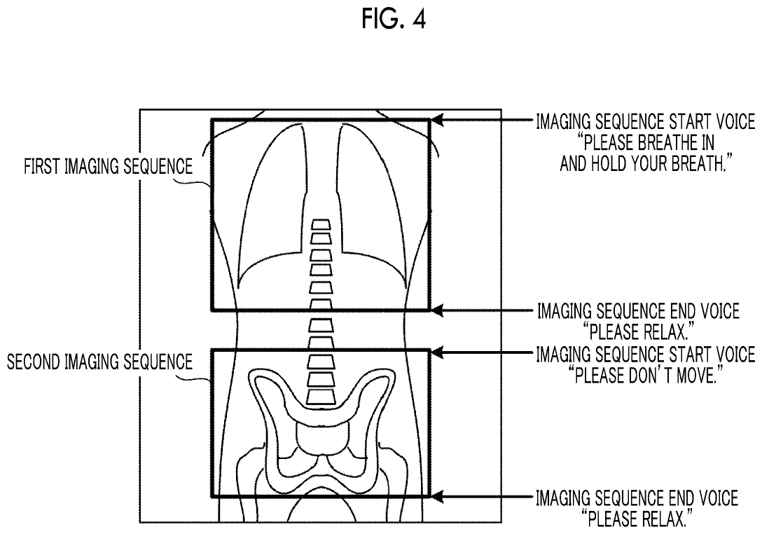

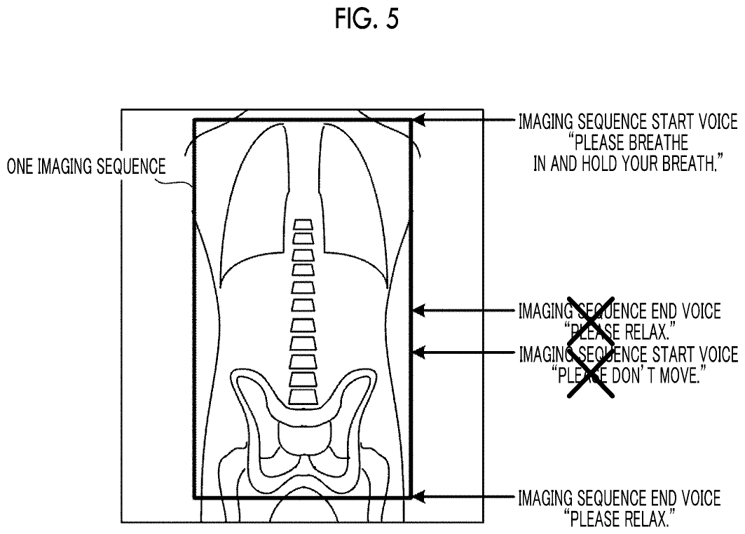

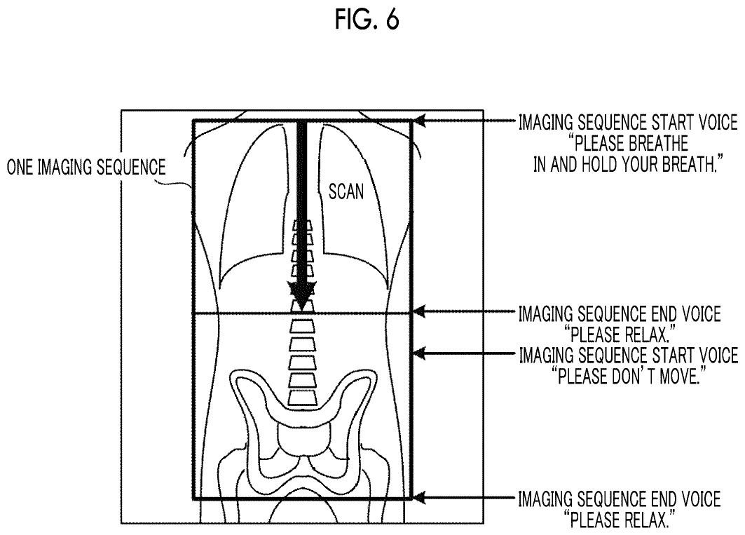

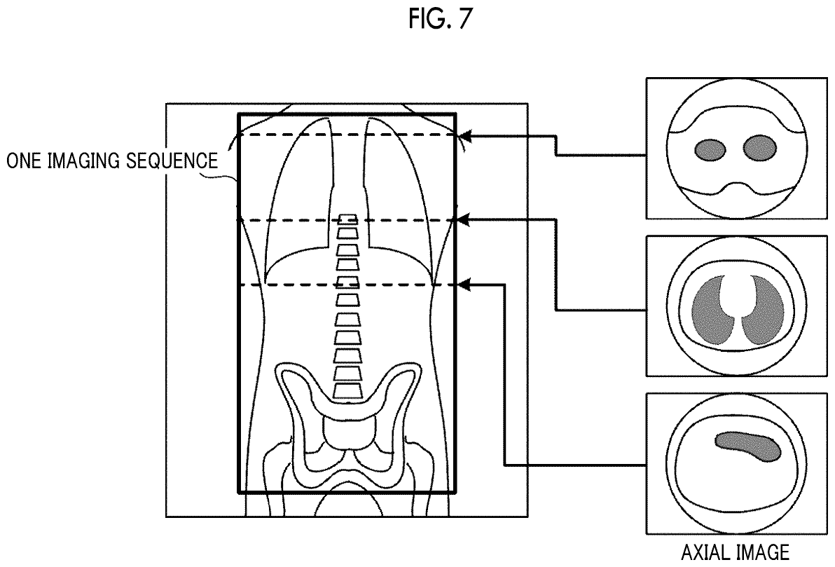

Next, processing of reproducing the voice guide according to the present embodiment will be described in detail with reference to FIGS. 4 to 7. FIG. 4 is a diagram showing a problem of the related art, and is a diagram showing an example of a reproduction state of the voice guide in a case in which a plurality of parts are imaged using a plurality of imaging sequences by a single continuous imaging operation. In addition, FIG. 5 is a diagram showing a problem of the related art, and is a diagram showing an example of a reproduction state of the voice guide in a case in which the plurality of parts are imaged using one imaging sequence by a single continuous imaging operation. In addition, FIG. 6 is a diagram showing the technology of the present disclosure, and is a diagram showing an example of the reproduction state of the voice guide in a case in which the plurality of parts are imaged using one imaging sequence by a single continuous imaging operation according to the technology of the present disclosure. Further, FIG. 7 is a diagram showing the technology of the present disclosure, and is a diagram showing an example of a reproduction method of the voice guide in a case in which the plurality of parts are imaged using one imaging sequence by a single continuous imaging operation according to the technology of the present disclosure.

As shown in FIG. 4 as an example, in the processing of reproducing the voice guide in the related art, in a case in which the plurality of parts such as the lung field and the lumbar vertebra are imaged by a single continuous imaging operation, the plurality of imaging sequences (in the example shown in FIG. 4, a “first imaging sequence” and a “second imaging sequence”) indicating a procedure of the imaging for each part that is the imaging target are set. In the processing of reproducing the voice guide in the related art, the voice guide is set in association with each imaging sequence. Therefore, in a case in which the voice guide such as “Please breathe in and hold your breath” or “Please relax” is provided for each part that is the imaging target while the imaging is being performed, the voice guide associated with the imaging sequence set for the corresponding part may be sequentially reproduced.

On the other hand, as shown in FIG. 5 as an example, in a case in which the plurality of parts are continuously imaged in a single imaging operation, it is desired that each part can be imaged in one imaging sequence.

However, in this case, although it is possible to reproduce the voice guide at the start or end of the imaging sequence, it is difficult to reproduce the voice guide related to the part, for the part to be imaged next, in the middle of the imaging sequence in an accurate period. This is because there is no trigger to reproduce the corresponding voice guide in the middle of the imaging sequence.

Therefore, as an example, as shown in FIGS. 6 and 7, during scanning with one imaging sequence corresponding to continuous imaging of the plurality of parts, the recognition unit 403A according to the present embodiment recognizes the imaging timings of the plurality of parts by the image recognition on the captured image (in the present embodiment, the axial image) obtained by the scanning. Then, the presentation unit 403B according to the present embodiment reproduces the corresponding voice guide at the recognized imaging timing.

In the present embodiment, the image recognition of the plurality of parts is performed using a learning model that has been trained in advance using the captured images as input information and information indicating the part imaged in the captured images as output information.

In the present embodiment, a model using a convolutional neural network (CNN) is applied as the learning model, but the present disclosure is not limited to this. For example, a form may be adopted in which, as the learning model, another model such as a recurrent neural network (RNN) model or an artificial intelligence (AI) model is applied.

In addition, the image recognition method of the plurality of parts is not limited to the method using the learning model described above, and for example, a method using a known pattern matching technique in the related art may be applied as the image recognition method of the plurality of parts.

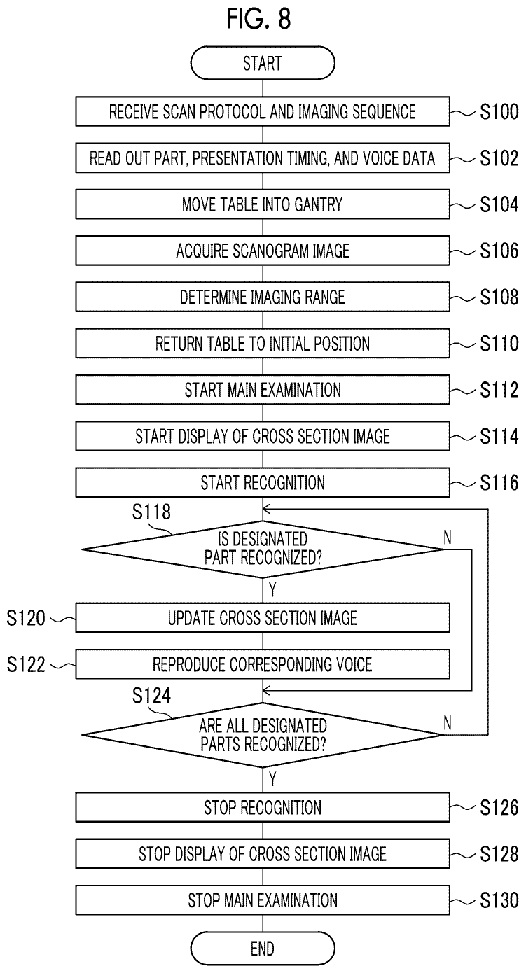

Hereinafter, an operation of the console 4 will be described with reference to FIG. 8.

FIG. 8 is a flowchart showing a flow of control of the medical diagnostic apparatus by the console 4. The CPU 41 reads out the control program of the medical diagnostic apparatus from the ROM 42 or the storage 44, and loads and executes the control program in the RAM 43, to perform control processing of the medical diagnostic apparatus. The flowchart shown in FIG. 8 is executed in a case in which the subject undergoes the examination with the medical diagnostic apparatus, in response to an instruction input by an imaging technician (hereinafter, referred to as a “user”) through the input unit 45.

In step S100, the CPU 41 receives a designation input of the scan protocol and the imaging sequence from the user.

After step S100, in step S102, the CPU 41 reads out each information of the part, the presentation timing, and the voice data corresponding to the scan protocol and the imaging sequence received from the user from the voice information database 53. Here, the read out part here is referred to as a “designated part” below.

After step S102, in step S104, the CPU 41 moves the table 3 into the gantry 11.

After step S104, in step S106, the CPU 41 irradiates the subject with the X-rays to acquire the scanogram image of the subject.

After step S106, in step S108, the CPU 41 determines the imaging range at the time of the main imaging as described above, using the acquired scanogram image.

After step S108, in step S110, the CPU 41 moves (returns) the table 3 to the initial position.

After step S110, in step S112, the CPU 41 starts the main examination on the subject by starting the imaging (main imaging) with the scanner 1 for the determined imaging range by starting the movement of the table 3 into the gantry 11.

After step S112, in step S114, the CPU 41 starts the display of the cross section image (in the present embodiment, the axial image, and hereinafter, simply referred to as the “cross section image”) corresponding to the captured image obtained by the imaging, by the display unit 46.

After step S114, in step S116, the CPU 41 starts the recognition of the designated part as the imaging target (examination target) by the above-described image recognition on the captured image (in the present embodiment, the axial image).

After step S116, in step S118, the CPU 41 determines whether the designated part is recognized, advances the processing to step S124 in a case in which a determination result is negative, and advances the processing to step S120 in a case in which the determination result is affirmative.

In step S120, the CPU 41 updates the cross section image such that the designated part recognized by the image recognition has a different state from the other parts.

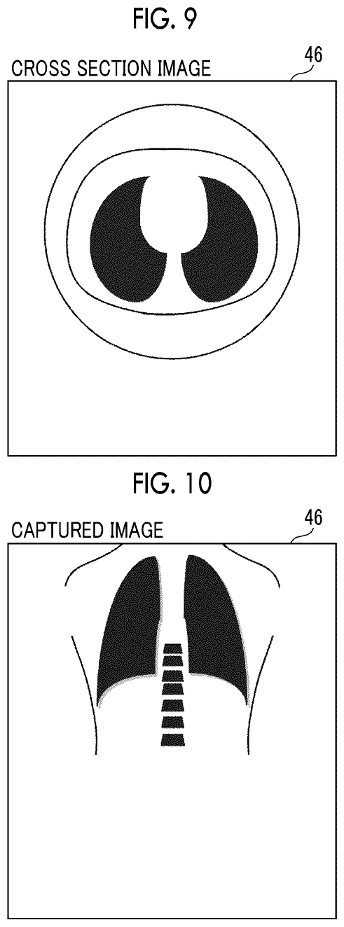

FIG. 9 is a diagram showing an example of a display state of the cross section image updated by the processing of step S120. As shown in FIG. 9, in the cross section image according to the present embodiment, the recognized designated part (in the example shown in FIG. 9, the lung field) is updated to a state (in the example shown in FIG. 9, a filled state) different from the other parts. Therefore, the user can determine whether there is an error in a recognition result by the image recognition by referring to the cross section image, and as a result, it is possible to prevent an error in the timing of the reproduction of the voice guide from occurring in advance.

After step S120, in step S122, the CPU 41 performs the control of reproducing the guide voice indicated by the voice data associated with the recognized designated part by the speaker 8 at the corresponding presentation timing.

After step S122, in step S124, the CPU 41 determines whether all the designated parts are recognized, returns the processing to step S118 in a case in which a determination result is negative, and advances the processing to step S126 in a case in which the determination result is affirmative.

By repeating the processing of step S118 to step S124, the display state of the cross section image is updated, and the corresponding voice guide is reproduced at the presentation timing for all the read out designated parts. In addition, a form may be adopted in which, in the repeated processing, from the recognized designated part, the imaging of the designated part to be subsequently included in the captured image is predicted, and the voice guide to be reproduced next is specified in advance using a prediction result.

In step S126, the CPU 41 stops the recognition of the designated part that is the imaging target started by the processing of step S116.

After step S126, in step S128, the CPU 41 stops the display of the cross section image started by the processing of step S114.

After step S128, in step S130, the CPU 41 stops the main examination started by the processing of step S112 and then ends the present control processing.

As described above, according to the embodiment of the present disclosure, the imaging period in which the part of the subject, which is associated with the presentation condition for presenting the predetermined instruction guide, is imaged is recognized from the captured images obtained in time series by imaging the subject, and the instruction guide is presented in accordance with the recognized imaging period. Therefore, even in a case in which imaging of the plurality of parts is performed by single continuous imaging, it is possible to present the instruction guide in an appropriate period.

In addition, according to the embodiment of the present disclosure, as the imaging period, the period including at least one of at least one of the start time of the imaging sequence indicating the procedure of the imaging, the end time of the imaging sequence, or the time point in the period after the start time and before the end time is applied. Therefore, it is possible to reproduce the instruction guide using the imaging sequence.

In addition, according to the embodiment of the present disclosure, it is determined whether the imaging period is likely to be approaching from the captured images, and the instruction guide is presented in accordance with the determined imaging period. Therefore, it is possible to present the instruction guide using a more appropriate imaging period as compared with a case in which it is not determined whether the imaging period is likely to be approaching.

In addition, according to the embodiment of the present disclosure, it is determined whether the imaging period is likely to be approaching by determining, from the part recognized by the image recognition on the captured image, whether another part to be subsequently included in the captured image is likely to be imaged. Accordingly, it is possible to determine the imaging period with higher accuracy than in a case of determining whether the imaging period is likely to be approaching, by using the imaging conditions.

In addition, according to the embodiment of the present disclosure, the cross section image corresponding to the captured image for which the imaging period is recognized is further presented such that the part recognized by the image recognition has a different state from the other parts. Therefore, it is possible to understand the result of the image recognition, and it is possible to prevent an erroneous operation caused by erroneous recognition in advance.

Further, according to the embodiment of the present disclosure, the voice guide by the voice is applied as the instruction guide, and the voice guide is reproduced in accordance with the recognized imaging period. Therefore, it is possible to understand the content of the guidance by the voice.

Although the embodiment of the present disclosure has been described in detail with reference to the accompanying drawings, the technical scope of the present disclosure is not limited to such an example. It is apparent that those having ordinary knowledge in the technical field of the present disclosure can conceive various change examples or modification examples within the scope of the technical idea described in the claims, and it is also understood that these change examples or modification examples belong to the technical scope of the present disclosure.

In addition, the effects described in the above-described embodiment are merely explanatory or exemplary examples, and the present disclosure is not limited to the effects described in the above-described embodiment. That is, the technology of the present disclosure can achieve other effects that are obvious to those having ordinary knowledge in the technical field of the present disclosure from the description of the above-described embodiment, in addition to the effects described in the above-described embodiment or instead of the effects described in the above-described embodiment.

For example, in the above-described embodiment, the information processing device according to the present disclosure is applied to the X-ray CT apparatus, but the present disclosure is not limited to this. In a case of an imaging apparatus that presents an instruction guide such as a voice guide to a target person, the information processing device according to the present disclosure may be applied to an MRI apparatus or the like.

In addition, in the above-described embodiment, a case has been described in which it is determined whether the imaging period (imaging timing in the above-described embodiment) is likely to be approaching, by the image recognition on the captured image, but the present disclosure is not limited to this. For example, a form may be adopted in which it is determined whether the imaging period is likely to be approaching, by using imaging conditions set in advance in accordance with the imaging of the subject.

For example, a form is described in which, as the imaging condition in this case, at least one of a scan speed of the scanner 1, a moving speed of the table 3, or a waiting time in a case in which there is a waiting time between the imaging of the plurality of parts that are the imaging targets in one imaging sequence is applied. In this case, for example, a form is described in which the imaging period of the part that is a voice guide reproduction target is estimated in accordance with an elapsed time from the start of the imaging for the imaging range by using the applied imaging conditions, and thus the imaging period in which the voice guide is reproduced is recognized. In this form, it is possible to determine whether the imaging period is likely to be approaching more easily than in a case of determining whether the imaging period is likely to be approaching by the image recognition.

In addition, in the above-described embodiment, as an example, a case has been described in which the cross section image shown in FIG. 9 is applied as the image for allowing the user to understand the error in the recognition result of the part that is the imaging target, but the present disclosure is not limited to this. For example, as shown in FIG. 10 as an example, a form may be adopted in which the captured image itself is applied as an image for allowing the user to understand the error in the recognition result of the part that is the imaging target. FIG. 10 is a diagram showing an example of a display state of the captured image according to the embodiment of the technology of the present disclosure.

In addition, in the above-described embodiment, a case has been described in which the imaging period is recognized using the captured image, but the present disclosure is not limited to this. For example, a form may be adopted in which the imaging period is recognized using the scanogram image. As a form in this case, for example, a form is described in which a region of the examination target part is specified from the scanogram image, and the instruction guide is presented for the specified region. Further, the flow of the control shown in the flowchart of FIG. 8, which is described in the above-described embodiment, is an example, and, for example, the processing of step S114 and the processing of step S116 may be interchanged, or the processing of step S126 and the processing of step S128 may be interchanged.

In the present embodiment, each processing is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for a specific use, a workstation, or another system that can execute each processing.

The processor may be configured by one or a plurality of types of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing, such as an application specific integrated circuit (ASIC), or hardware such as a graphic processing unit (GPU) or a neural processing unit (NPU). Furthermore, the types of hardware may be a combination of different types of hardware. In a case in which a plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. In addition, hardware is implemented in a form of an electric circuit (circuitry) in which circuit elements, such as semiconductor elements, are combined.

Furthermore, the program may be software, such as firmware or a microcode. Further, the program may be, for example, a program module group, and each function thereof may be implemented by a processor configured to execute each function. The program may be a program code or a plurality of code segments stored in one or a plurality of non-transitory computer-readable media (for example, a storage medium and another storage). The program may be stored in the plurality of non-transitory computer-readable media existing in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or content of the memory.

In addition, in the above-described embodiment, the aspect has been described in which the information processing program is stored (installed) in advance in the ROM 42 or the storage 44 of the console 4, but the present disclosure is not limited to this. The information processing program may be provided in a form of being recorded in a recording medium such as a compact disc read-only memory (CD-ROM), a digital versatile disc read-only memory (DVD-ROM), and a universal serial bus (USB) memory. Furthermore, an aspect may be adopted in which the information processing program is downloaded from an external device via a network.

The technology of the present disclosure extends to a computer-readable storage medium (CD-ROM, DVD-ROM, USB memory, or the like) that non-transitorily stores the information processing program, in addition to the information processing program.

Furthermore, the present invention can also be applied to a program and a program product.

In regard to the above-described embodiment, the following supplementary notes will be further disclosed.

### Supplementary Note 1

An information processing device comprising: a processor, in which the processor recognizes an imaging period in which a part of a subject, which is associated with a presentation condition for presenting a predetermined instruction guide, is imaged from captured images obtained in time series by imaging the subject, and presents the instruction guide in accordance with the recognized imaging period.

### Supplementary Note 2

The information processing device according to supplementary note 1, in which the imaging period is a period including at least one of a start time of an imaging sequence indicating a procedure of the imaging, an end time of the imaging sequence, or any time point in a period after the start time and before the end time.

### Supplementary Note 3

The information processing device according to supplementary note 1 or 2, in which the processor determines whether the imaging period is likely to be approaching from the captured images, and presents the instruction guide in accordance with the determined imaging period.

### Supplementary Note 4

The information processing device according to supplementary note 3, in which the processor determines whether the imaging period is likely to be approaching by using imaging conditions set in advance in correspondence with the imaging for the subject.

### Supplementary Note 5

The information processing device according to supplementary note 3, in which the processor determines whether the imaging period is likely to be approaching by determining, from the part recognized by image recognition on the captured image, whether another part to be subsequently included in the captured image is likely to be imaged.

### Supplementary Note 6

The information processing device according to supplementary note 5, in which the processor further presents a cross section image corresponding to the captured image for which the imaging period is recognized, such that the part recognized by the image recognition has a different state from other parts.

### Supplementary Note 7

The information processing device according to any one of supplementary notes 1 to 6, in which the instruction guide is a voice guide by voice, and the processor reproduces the voice guide in accordance with the recognized imaging period.

### Supplementary Note 8

The information processing device according to any one of supplementary notes 1 to 7, in which the instruction guide is a display guide by display, and the processor displays the display guide in accordance with the recognized imaging period.

### Supplementary Note 9

A medical imaging apparatus comprising: the information processing device according to any one of supplementary notes 1 to 8; and an imaging device that performs main imaging for a medical image while the instruction guide is presented by the information processing device.

### Supplementary Note 10

An information processing method executed by a processor, comprising: recognizing an imaging period in which a part of a subject, which is associated with a presentation condition for presenting a predetermined instruction guide, is imaged from captured images obtained in time series by imaging the subject; and presenting the instruction guide in accordance with the recognized imaging period.

### Supplementary Note 11

An information processing program causing a computer to execute a process comprising: recognizing an imaging period in which a part of a subject, which is associated with a presentation condition for presenting a predetermined instruction guide, is imaged from captured images obtained in time series by imaging the subject; and presenting the instruction guide in accordance with the recognized imaging period.

What is claimed is:

1. An information processing device comprising:
a processor is configured to,
recognize an imaging period during which a part of a subject is imaged in time series of captured images by imaging the subject, wherein the part is associated with a presentation condition for presenting a predetermined instruction guide, and
present the instruction guide in accordance with the imaging period.

2. The information processing device according to claim 1,
wherein the imaging period is a period including at least one of a start time of an imaging sequence indicating a procedure of the imaging, an end time of the imaging sequence, or any time point in a period after the start time and before the end time.

3. The information processing device according to claim 1,
the processor is further configured to
determine whether the imaging period is likely to be approaching from the captured images, and
present the instruction guide in accordance with the determined imaging period.

4. The information processing device according to claim 2,
the processor is further configured to
determine whether the imaging period is likely to be approaching from the captured images, and
present the instruction guide in accordance with the determined imaging period.

5. The information processing device according to claim 3,
the processor is further configured to determine whether the imaging period is likely to be approaching by using imaging conditions set in advance in correspondence with the imaging for the subject.

6. The information processing device according to claim 4,
wherein the processor determines whether the imaging period is likely to be approaching by using imaging conditions set in advance in correspondence with the imaging for the subject.

7. The information processing device according to claim 3,
the processor is further configured to
determine, from at least one part which is recognized by image recognition on at least one of the captured image, whether the part, associated with the presentation condition, to be subsequently included in the captured image is likely to be imaged.

8. The information processing device according to claim 4,
wherein the processor determines whether the imaging period is likely to be approaching by determining, from the part recognized by image recognition on the captured image, whether another part to be subsequently included in the captured image is likely to be imaged.

9. The information processing device according to claim 7,
the processor is further configured to present a cross section image corresponding to the captured image during the imaging period, such that the part has a different state from other parts.

10. The information processing device according to claim 8,
wherein the processor further presents a cross section image corresponding to the captured image for which the imaging period is recognized, such that the part recognized by the image recognition has a different state from other parts.

11. The information processing device according to claim 1,
wherein the instruction guide is a voice guide, and
the processor reproduces the voice guide in accordance with the imaging period.

12. The information processing device according to claim 2,
wherein the instruction guide is a voice guide by voice, and
the processor reproduces the voice guide in accordance with the imaging period.

13. The information processing device according to claim 1,
wherein the instruction guide is a display guide on a display, and
the processor displays the display guide in accordance with the imaging period.

14. The information processing device according to claim 2,
wherein the instruction guide is a display guide by display, and
the processor displays the display guide in accordance with the imaging period.

15. A medical imaging apparatus comprising:
the information processing device according to claim 1; and
an imaging device that performs main imaging for a medical image while the instruction guide is presented by the information processing device.

16. An information processing method executed by a processor, comprising:
recognizing an imaging period during which a part of a subject is imaged in time series of captured images by imaging the subject, wherein the part is associated with a presentation condition for presenting a predetermined instruction guide; and
presenting the instruction guide in accordance with the recognized imaging period.

17. A non-transitory computer-readable storage medium storing an information processing program causing a computer to execute a process comprising:
recognizing an imaging period during which a part of a subject is imaged in time series of captured images by imaging the subject, wherein the part is associated with a presentation condition for presenting a predetermined instruction guide; and
presenting the instruction guide in accordance with the recognized imaging period.
